(19) United States
(12) Patent Application Publication
(10) Pub. No.: US 2026/0355258 A1
(43) Pub. Date: Nov. 12, 2026

(54) APPLICATION OF TESTUDINIS CARAPAX ET PLASTRUM IN TREATING FEMALE SEXUAL DYSFUNCTION

(71) Applicant: Shengtian Wang, Tianjin (CN)

(72) Inventors: Shengtian Wang, Tianjin (CN); Zheng Wang, Tianjin (CN)

(21) Appl. No.: 19/238,942

(22) Filed: Jun. 16, 2025

(30) Foreign Application Priority Data
May 12, 2025 (CN) 202510606001.9

(51) Int. Cl. A61K 35/586; A61K 47/36; A61K 47/38; A61P 15/02

(52) U.S. Cl. CPC A61K 35/586; A61K 47/36; A61K 47/38; A61P 15/02

(57) ABSTRACT

An application of Testudinis Carapax et Plastrum in treating female sexual dysfunction. The invention for the first time applies Testudinis Carapax et Plastrum to treat sexual dysfunction in women, particularly menopausal women, with remarkable therapeutic effects, no side effects, simple composition, and low cost. Therefore, the invention provides new directions and more choices for improving and treating female sexual dysfunction.

### TECHNICAL FIELD

The invention belongs to the field of medical technology, specifically relates to an application of Testudinis Carapax et Plastrum in treating female sexual dysfunction.

### BACKGROUND ART

Female sexual dysfunction (FSD) refers to disorders in one or more stages of the sexual intercourse process in women, including lack of sexual desire, inability to arouse sexual desire, failure to achieve orgasm, or symptoms such as pain related to sexual intercourse. FSD predominantly affects women around menopause. The physical and psychological distress caused by FSD impacts quality of life and even family harmony. However, current societal attention to FSD remains low. Few effective medications exist for treating FSD, among which hormonal drugs show better efficacy but have significant side effects.

### SUMMARY OF THE INVENTION

Against this background, the invention provides an application of Testudinis Carapax et Plastrum in treating female sexual dysfunction. Using Testudinis Carapax et Plastrum as an active component, it can effectively improve and treat sexual dysfunction in women, particularly menopausal women.

The technical solution provided by the invention is:

An application of Testudinis Carapax et Plastrum in preparing medications for treating female sexual dysfunction.

Further, the female sexual dysfunction is characterized by at least one of the following:
\textcircled{1} reduced sexual desire or complete lack of sexual desire;
\textcircled{2} sexual arousal disorders and/or insufficient lubrication;
\textcircled{3} difficulty achieving and maintaining lubrication;
\textcircled{4} difficulty achieving orgasm, low orgasm intensity, or absence of sexual pleasure;
\textcircled{5} pain during sexual intercourse.

Further, the women referred to are menopausal women.

Further, the menopausal women are perimenopausal women.

Further, the medication uses extracts of Testudinis Carapax et Plastrum as an active ingredient.

Further, the medication can be formulated into vaginal administration preparations with medically acceptable adjuvants.

Specifically, it can be solid preparations such as tablets, capsules, powders, granules, dropping pills, suppositories; or liquid preparations such as sols, suspensions, and emulsions.

Based on the same inventive concept, the invention also provides an application of Testudinis Carapax et Plastrum in preparing products for enhancing female sexual arousal and/or sexual desire.

Further, the product is a vaginal administration preparation.

Based on the same inventive concept, the invention also provides a medication for treating female sexual dysfunction, which is prepared from the following raw materials by weight:
70-85% Testudinis Carapax et Plastrum Colla, 2-15% microcrystalline cellulose, and 5-15% pregelatinized starch.

Further, the medication is a vaginal administration capsule.

Beneficial effects achieved by the invention is as follows:
the invention for the first time applies Testudinis Carapax et Plastrum to treat sexual dysfunction in women, particularly menopausal women, with remarkable therapeutic effects, no side effects, simple composition, and low cost. Therefore, the invention provides new directions and more choices for improving and treating female sexual dysfunction.

### SPECIFIC EMBODIMENT OF THE INVENTION

To make the objectives, technical solutions, and advantages of the embodiments of the invention clearer, the technical solutions in the embodiments of the invention will be described clearly and completely below. Obviously, the described embodiments are some, but not all, embodiments of the invention. All other embodiments obtained by those of ordinary skill in the art without creative efforts based on the embodiments of the invention fall within the protection scope of the present invention.

Embodiment 1: a medication for treating female sexual dysfunction is prepared from the following raw materials by weight:
82% Testudinis Carapax et Plastrum Colla, 6% microcrystalline cellulose, and 12% pregelatinized starch.

The Testudinis Carapax et Plastrum Colla in this embodiment can be extracted using water or ethanol as a solvent and concentrated into gelatin.

Specifically, when using water as the solvent, the preparation of the medication includes the following steps:
1) take clear Testudinis Carapax et Plastrum, cut and crush it into appropriately sized chunks or slices;
2) after crushing the Testudinis Carapax et Plastrum, add water until the Testudinis Carapax et Plastrum is fully submerged, then slowly decoct over low to medium heat for 30-45 minutes; add boiling water as needed to supplement evaporated water. After extracting the decoction, add water again to the Testudinis Carapax et Plastrum and decoct for another 30-45 minutes. Repeat this process once more to obtain the decoction for the third time;
3) combine three decoctions, filter, and retain the filtrate;
4) heat and concentrate the filtrate to collect gelatin until it reaches the “hanging flag” stage (i.e., when scooped with a spoon, the gelatin solution drips down along an edge of the spoon in a flag-like shape). Stop heating, wait until the temperature drops to 60 °C, add vitamin E while stirring, and after complete solidification, vacuum-dry, crush, and sieve through a 50-80 mesh sieve. Mix the resulting powder with microcrystalline cellulose and talcum powder;
5) fill the above medication into 0.5 g capsules made of gelatin.

### Embodiment 2

Thirty-six female patients with female sexual dysfunction (evaluated by a Female Sexual Function Index (FSFI) score <25) were enrolled, aged 42-57 years, including 5 who had been postmenopausal for over 1 year and 26 who were determined by doctors to be in the perimenopausal period.

The FSFI is a widely used multidimensional standardized tool for assessing female sexual dysfunction (FSD), covering six dimensions: sexual desire, sexual arousal, vaginal lubrication, orgasm, satisfaction, and pain during intercourse. The total score ranges from 0 to 36, with higher scores indicating better sexual function.

Exclusion criteria: \textcircled{1} pregnancy; \textcircled{2} urinary and genital infections, malignant tumors, within 1 month after gynecological surgery, severe organic diseases of the heart, liver, or kidney, neurological diseases, intellectual or mental disorders; \textcircled{3} sexual dysfunction caused by gynecological inflammation, organic lesions, or endocrine system diseases; \textcircled{4} sexual dysfunction caused by psychological factors; \textcircled{5} caused by male partner factors; \textcircled{6} lack of a stable sexual partner; \textcircled{7} taking medications that affect or are suspected to affect sexual function (e.g., antihypertensives, sedatives).

The enrolled patients were treated with the medication from Embodiment 1. One capsule was inserted into the vagina near the cervix before bedtime daily for 16 consecutive weeks, after which FSFI evaluation was performed again.

### Efficacy Criteria:

cure: FSFI score >25;
effective: FSFI score increased more than 10 points (including 10 points) but less than or equal to 25;
ineffective: no change or decrease in FSFI score, or an increase of less than 10 points.

Results: 4 cases were ineffective, 12 cases were effective, 20 cases showed marked efficacy, with an effective rate of 88.9%.

Comparison of sexual function indexes (average values) before and after intervention in these patients is shown in the table below:

| FSFI | | | Vaginal | | | | |
|---|---|---|---|---|---|---|---|
| Dimension | Sexual | Sexual | Lubri- | | Satis- | | Total |
| (Points) | Desire | Arousal | cation | Orgasm | faction | Pain | Score |
| Before | 2.59 | 2.14 | 3.52 | 3.41 | 3.79 | 3.83 | 19.28 |
| Treatment | | | | | | | |
| After | 3.87 | 3.96 | 4.05 | 4.33 | 4.68 | 4.52 | 25.41 |
| Treatment | | | | | | | |

The present invention can also be used to enhance sexual desire and pleasure in normal women. For example, inserting one capsule into the vagina near the cervix 20-30 minutes before sexual activity can promote excitement, facilitate orgasm, and enhance sexual pleasure.

The invention and its embodiments have been described above, but the description is not limited thereto; only one embodiment of the invention is shown in the drawings, and the actual structure is not limited thereto. In general, it is to be understood by those skilled in the art that non-creative design of structural forms and embodiments that are similar to the technical solutions without departing from the spirit of the invention shall all fall within the protective scope of the invention.

1. An application of Testudinis Carapax et Plastrum in preparing medications for treating female sexual dysfunction.

2. The application according to claim 1, wherein the female sexual dysfunction is characterized by at least one of the following:
\textcircled{1} reduced sexual desire or complete lack of sexual desire;
\textcircled{2} sexual arousal disorders and/or insufficient lubrication;
\textcircled{3} difficulty achieving and maintaining lubrication;
\textcircled{4} difficulty achieving orgasm, low orgasm intensity, or absence of sexual pleasure;
\textcircled{5} pain during sexual intercourse.

3. The application according to claim 2, wherein the women referred to are menopausal women.

4. The application according to claim 3, wherein the menopausal women are perimenopausal women.

5. The application according to claim 1, wherein the medication uses extracts of Testudinis Carapax et Plastrum as an active ingredient.

6. The application according to claim 1, wherein the medication can be formulated into vaginal administration preparations with medically acceptable adjuvants.

7. An application of Testudinis Carapax et Plastrum in preparing products for enhancing female sexual arousal and/or sexual desire.

8. The application according to claim 7, wherein the product is a vaginal administration preparation.

9. A medication for treating female sexual dysfunction, which is prepared from the following raw materials by weight:
70-85% Testudinis Carapax et Plastrum Colla, 2-15% microcrystalline cellulose, and 5-15% pregelatinized starch.

10. The medication for treating female sexual dysfunction according to claim 9, wherein the medication is a vaginal administration capsule.
